(19) United States
(12) Patent Application Publication
(10) Pub. No.: US 2018/0098813 A1
(43) Pub. Date: Apr. 12, 2018

(54) METHOD AND SYSTEM FOR RENDERING A MEDICAL SIMULATION IN AN OPERATING ROOM IN VIRTUAL REALITY OR AUGMENTED REALITY ENVIRONMENT

(71) Applicant: SIMBIONIX LTD., Airport City (IL)

(72) Inventors: Lior NESICHI, Rehovot (IL); Mordechai ZASLAVSKI, Bet-Shemesh (IL); Eran NEGRIN, Rishon LeZion (IL); Niv FISHER, Ramat Gan (IL); Sophia SLEPNEV, Tel Aviv-Jaffa (IL)

(73) Assignee: SIMBIONIX LTD., Airport City (IL)

(21) Appl. No.: 15/720,629

(22) Filed: Sep. 29, 2017

Related U.S. Application Data
(60) Provisional application No. 62/405,367, filed on Oct. 7, 2016.

(51) Int. Cl. A61B 34/10; G09B 23/28; G09B 5/06

(52) U.S. Cl. CPC A61B 34/10; G09B 23/28; A61B 2034/105; A61B 2034/102; G09B 5/065

(57) ABSTRACT

Systems and methods for rendering medical procedures in a virtual reality operating room for a training a trainee are provided. A medical procedure can be simulated and a trainee can manually manipulate a medical tool to perform the simulated medical procedure in virtual reality on a virtual reality avatar in the virtual reality simulation.

### CROSS REFERENCE TO RELATED APPLICATION

This patent application claims benefit and priority from co-pending U.S. Provisional Patent Application 62/405,367 filed on Oct. 7, 2016, the entire contents which is hereby incorporated by reference in its entirety.

### FIELD OF THE INVENTION

The invention relates generally to medical simulations used to train medical personnel. In particular, the invention relates to rendering a medical simulation in a virtual reality or augmented reality environment that present an operating room experience to a trainee.

### BACKGROUND OF THE INVENTION

Currently, medical simulators can be used to train medical personnel. For example, a trainee (e.g., doctor) can use a computer to perform a computer simulated surgery. The computer simulated surgery can include a display screen that displays images appropriate for a particular surgery, and tools (e.g., haptic tools) that the trainee can manipulate to simulate a surgical experience.

For example, assume a doctor desires to simulate operating on a clogged heart artery of a patient. The doctor can have a set of haptic tools that correspond to the tools that a doctor uses in a real surgery. The doctor selects on a computing device a simulation that corresponds to the clogged heart surgery. The computing device displays on a screen skin of a patient. The doctor can then use one tool of the set of tools to cut the patient open by manually manipulating the haptic tool. The tool can include sensors that senses the doctors manual manipulation, sends the information to the computer simulation and the computer simulation can display the images that correspond to the movements sensed by the tools. Some common tools used for medical simulations include endoscopes, laparoscopes, and/or other operating room machinery.

One difficulty with current simulators is that they typically do not provide a trainee with a realistic experience of being in an operating room. During real operations, there can be many distractions for the surgeon and other medical personnel. For example, a surgeon can be called on an overhead calling system. A nurse can drop a tool being passed to the doctor at a crucial moment. Current medical training simulators can be limited in that they typically do not provide the trainee with a realistic experience.

A virtual reality (VR) medical simulation may be extremely performance intensive, and may take a very heavy toll on both the Graphics Processing Unit (GPU) and a personal computer (PC) that includes a Central Processing Unit (CPU).

The trainee (e.g., the person who is using the medical simulation) may wear 3 dimensional (3D) VR glasses or augment reality (AR) glasses. Typically, in VR, to provide a realistic experience to the user and/or trainee through the 3D VR glasses, a rendering refresh rate of at least 90 HZ, and a recommended refresh rate of 120 HZ is required. Lower refresh rates can cause hardware latency that is noticeable to a trainee by causing, for example, a time gap which can be considerably longer than what is typically required for the trainee to experience an uninterrupted 3D VR experience. The latency can refer to a length of time passing between trainee's head movement, detection of this head movement by VR sensor(s), and the following and corresponding response in the VR glasses—which can translate the head movement into a new viewing angle within a VR scene that is displayed to the trainee.

For example, a sharp turn of the head from a straight-forward look to glance to the left-hand side may take a fraction of a second too long, until the VR glasses view is updated from a head-on view to a view of the left-hand side of the VR environment. In VR, small of latencies can cause the trainee dizziness, loss of focus, severe headaches and nausea. A VR resolution of approximately 1000×1000 pixels or higher per eye is typically recommended. A lower resolution can cause a pixelated view, aliasing and/or a loss of ‘suspension of disbelief’ on part of the viewer.

Therefore, it can be desirable to provide a trainee with a realistic experience. It can also be desirable to provide a VR simulation of a medical procedure that complies with the rendering rate and resolution that can allow a user to have a realistic experience sufficient for medical training.

### SUMMARY OF THE INVENTION

One advantage of the invention can include providing a trainee with a real-life experience via performing a medical procedure simulation in a virtual reality or augmented reality operating room. Another advantage of the invention can include an ability to render a VR/AR scene based on a haptic medical procedure simulator.

In one aspect, the invention involves a system for rendering medical procedures in a virtual reality operating room for training a trainee. The system includes a user input device for the trainee to select a type of medical procedure to simulate. The system also includes a haptic medical tool for the trainee to manually manipulate during the simulation. The system also includes a haptics medical simulation system for rendering the simulation of the selected medical procedure at a first predetermined frame rate based on the haptic medical tool manipulation. The system also includes a virtual reality simulation system coupled to the medical procedure simulation system to render i) a virtual reality operating room scene that corresponds to the type of medical procedure to simulate, and ii) the simulation of the selected medical procedure into a virtual reality scene. The system also includes a surface sharing module coupled to the haptics medical simulation system and the virtual reality simulation system, the surface sharing module providing simulation information from the simulation system to the virtual reality simulation system that allows the virtual reality simulation system to render the virtual reality operating room scene that corresponds to the haptics medical simulation. The system also includes a virtual reality headset coupled to the virtual reality simulation system for the trainee to view the virtual reality scene.

In some embodiments, the simulation information comprises x-ray, Ultrasound, magnetic resonance imaging, CT scan, monitor haptic simulation information, anatomy, vital signs or any combination thereof. In some embodiments, the virtual reality simulation system modifies the simulation information from the surface sharing module with post processing effects.

In some embodiments, the post processing effects comprises modifying visual appearance of rendered object that correspond to the simulation information based on the environment in the virtual reality operating room scene. In some embodiments, the virtual reality simulation system renders the virtual reality scene with a priority over the haptics medical simulation system.

In some embodiments, the virtual reality simulation system renders at a rate of at least 90 frames per second. In some embodiments, the haptics medical simulation renders at a rate of the virtual reality simulation system. In some embodiments, the haptics medical simulation system and the virtual reality simulation system are rendering the medical simulation in parallel.

In another aspect, the invention includes a method for rendering medical procedures in a virtual reality operating room for training a trainee. The method includes selecting a medical procedure to simulate a user input device for the trainee to select a type of medical procedure to simulate. The method also involves receiving haptic input from a haptic medical tool for the trainee to manually manipulate during the simulation. The method also involves rendering, via a haptics medical simulation system, the simulation of the selected medical procedure at a first predetermined frame rate based on the haptic medical tool manipulation. The method also involves rendering, via a virtual reality simulation system coupled to the medical procedure simulation system, i) a virtual reality operating room scene that corresponds to the type of medical procedure to simulate, and ii) the simulation of the selected medical procedure into a virtual reality scene. The method also involves providing simulation information, via a surface sharing module, from the medical simulation system to the virtual reality simulation system that allows the virtual reality simulation system to render the virtual reality operating room scene that corresponds to the haptics medical simulation. The method also involves displaying, via a virtual reality headset, the virtual reality scene.

In some embodiments, the simulation information comprises x-ray, Ultrasound, magnetic resonance imaging, CT scan, monitor haptic simulation information, anatomy, vital signs or any combination thereof. In some embodiments, the method involves modifying, via the virtual reality simulation system, the simulation information from the surface sharing module with post processing effects.

In some embodiments, the post processing effects comprises modifying visual appearance of rendered object that correspond to the simulation information based on the environment in the virtual reality operating room scene. In some embodiments, the virtual reality simulation system renders the virtual reality scene with a priority over the haptics medical simulation system.

In some embodiments, the virtual reality simulation system renders at a rate of at least 90 frames per second. In some embodiments, the haptics medical simulation renders at a rate of the virtual reality simulation system. In some embodiments, the haptics medical simulation system and the virtual reality simulation system are rendering the medical simulation in parallel.

### BRIEF DESCRIPTION OF THE DRAWINGS

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature of an embodiment of the disclosure in a figure can be used to reference the given feature. Dimensions of features shown in the figures are chosen for convenience and clarity of presentation and are not necessarily shown to scale.

The subject matter regarded as the invention is particularly pointed out and distinctly claimed in the concluding portion of the specification. The invention, however, both as to organization and method of operation, together with objects, features and advantages thereof, can best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 shows a block diagram of a system for simulating medical procedures in a virtual reality operating room for training a trainee, according to an illustrative embodiment of the invention;

FIG. 2 is a flow chart for a method for rendering via the medical procedure simulation module of FIG. 1, according to an illustrative embodiment of the invention.

FIG. 3 is a flow chart for a method for rendering via the VR simulation system of FIG. 1, according to an illustrative embodiment of the invention;

FIG. 4 shows a flow chart of a method for simulating medical procedures in a virtual reality operating room for training a trainee, according to an illustrative embodiment of the invention; and

FIGS. 5a-5f are diagrams showing examples of a trainee using the simulation system of FIG. 1, 2, 3 or the method of FIG. 4, according to illustrative embodiments of the invention.

It will be appreciated that for simplicity and clarity of illustration, elements shown in the figures have not necessarily been drawn accurately or to scale. For example, the dimensions of some of the elements can be exaggerated relative to other elements for clarity, or several physical components can be included in one functional block or element. Further, where considered appropriate, reference numerals can be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the invention can be practiced without these specific details. In other instances, well-known methods, procedures, and components, modules, units and/or circuits have not been described in detail so as not to obscure the invention. Some features or elements described with respect to one embodiment can be combined with features or elements described with respect to other embodiments. For the sake of clarity, discussion of same or similar features or elements cannot be repeated.

Although embodiments of the invention are not limited in this regard, discussions utilizing terms such as, for example, “processing,” “computing,” “calculating,” “determining,” “establishing”, “analyzing”, “checking”, or the like, can refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that can store instructions to perform operations and/or processes. Although embodiments of the invention are not limited in this regard, the terms “plurality” and “a plurality” as used herein can include, for example, “multiple” or “two or more”. The terms “plurality” or “a plurality” can be used throughout the specification to describe two or more components, devices, elements, units, parameters, or the like. The term set when used herein can include one or more items. Unless explicitly stated, the method embodiments described herein are not constrained to a particular order or sequence. Additionally, some of the described method embodiments or elements thereof can occur or be performed simultaneously, at the same point in time, or concurrently.

In general, a system is provided that can allow for a medical simulator to provide a medical procedure simulation to a trainee in virtual reality. The system can include a medical procedure simulation system that communicates with a virtual reality (VR) simulation system. The trainee (e.g., user) can interact with the medical simulator via one or more medical (e.g., surgical) tools, and experience the medical simulation in virtual reality (or augmented reality). The VR simulation system can use a headset/glasses to present a VR operating room scene to the trainee. The VR operating room scene can include an operating table, vital sign monitors, and/or any equipment that can be present in a real life operating room.

A patient (e.g., avatar or bot) to be operated on can also appear in the operating room scene. The medical procedure simulation system can receive inputs from the medical tools, and as the medical procedure simulation system runs the simulation, the medical procedure simulation information can be used by the VR simulation system to render the medical procedure simulation onto the VR patient. The patient can respond to the trainee's manipulation of the one or more surgical tools during the simulation.

In general, the system can accommodate multiple trainees in one simulation. For example, a surgeon trainee can experience a simulation of a heart surgery simulation on a patient (e.g., avatar) and a nurse trainee can assist the surgeon trainee. The surgeon can see the nurse trainee depicted as a bot within the VR scene, and the nurse trainee can see the surgeon depicted as bot within the VR scene. The surgeon trainee can use haptic tools associated with the medical procedure simulator, and during the simulation, the nurse trainee can pass the surgeon trainee virtual tools in the VR scene.

The medical simulation system and the virtual reality simulation system can each render the medical simulation in parallel. The medical simulation system can share its information with the virtual reality simulation system such that the medical simulation system is not effected by the medical simulation system.

In some embodiments, an augmented reality (AR) simulation system is used instead of a VR simulation system. In these embodiments, an AR scene is presented to the trainee. The AR scene can include any objects that are typically found in a real-life operating room.

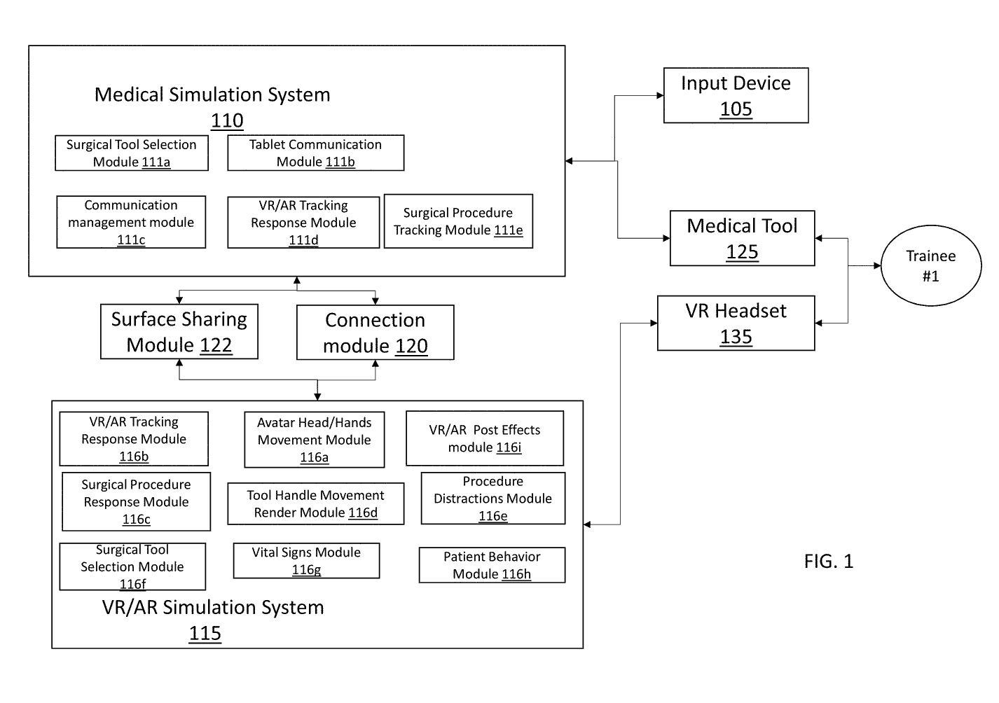

FIG. 1 shows a block diagram of a system for simulating medical procedures in a virtual reality (or augmented reality) operating room for training a trainee, according to an illustrative embodiment of the invention. The system includes an input device 105, a medical procedure simulation system 110, a virtual reality and/or augmented reality (VR/AR) simulation system 115, a connection module 120, a surface sharing module 122, a medical tool 125, a virtual reality headset 135. For the purpose of simplicity, the discussion with respect to FIG. 1 will focus on a VR simulation system. However, as is apparent to one of ordinary skill in the art, the simulation system can be virtual reality or augmented reality.

The connection module 120 can include allow for information to flow between the VR simulation system 115 and the medical procedure simulation system 110. For example, inputs received by VR simulation system 115 and/or modifications to the VR operating room scene can be provided to the connection module 120. Inputs received by the medical procedure simulation system 110 and/or medical procedure status information can be provided to the connection module 120. The connection module 120 can be a memory mapped file, one or more pipes, TCP/IP socket communication channels or any combination thereof.

The surface sharing module 122 can allow for surfaces rendered by the medical procedure simulation system 110 to be shared with the VR simulation system 115.

The medical procedure simulation system 110 can be coupled to the input device 105, the medical tool 125 to receive one or more inputs. The medical tool can be a device that can sense motion and touch of the trainee. The medical tool 125 can be a device that is capable of intaking haptic inputs. For example, the medical tool 125 can be a laparoscopic trocar or GI/Bronchoscopy tools. The input device 105 can be a tablet, smart phone, personal computer, touch screen device, or any combination thereof.

The medical procedure simulation system 110 can also be coupled to the VR simulation system 115 via the connection module 120. The medical procedure simulation system 110 can include a central processing unit and/or a graphics processing unit. The medical procedure simulation system 110 can simulate medical procedures as shown, for example, in U.S. Pat. No. 7,850,456, which is incorporated herein by reference in its entirety.

The medical procedure simulation system 110 can include a surgical tool selection module 111a, a tablet communication module 111b, a communication management module 111c, a virtual reality (VR)/augmented reality (AR) tracking response module 111d, and/or a surgical procedure tracking module 111e.

The tablet communication module 111b can receive input from a trainee. For example, a trainee can select a particular medical procedure to simulate and/or specify a number of participants in the simulation. In some embodiments, a proctor overseeing the training can add to the simulation and receive information from the simulation via the tablet. For example, the proctor can input an injury, and simulation can display to the proctor via the table status (e.g., vessel structure status and/or when the injury is controlled or uncontrolled).

The surgical tool selection module 111a can determine one or more surgical tools (e.g., haptic tools or virtual tools) that can be used in the medical procedure simulation based on the selected medical procedure. The one or more surgical tools can be virtual or haptic tools. The surgical tool selection module 111a can also determine which surgical tools can be available in the simulation based on potential tool entry points on the avatar being operated on in the simulation. For example, tool entry points of trocars, open incisions, and/or body cavities. For example, an arterial point of entry for a stent or catheter, can indicate that a laparoscopic trocar can be available. In another example, for an ultrasound simulation an ultrasound probe can be made available. The surgical tool selection module 111a can include a surgical stool status of one or more surgical tools in the medical simulation. For example, a surgical tool status of whether any tool is currently inside/outside of the patient body, whether the particular tool was selected for a particular entry point, a position of the tool, an orientation of the tool and/or properties of the tool (e.g., type and/or name). The surgical tool selection module 111a can also receive surgical tools status information from the VR/AR simulation system 1115. For example, a changed of surgical tool during the medical procedure where the change is from a haptic tool to a virtual tool.

The VR/AR tracking response module 111d can modify the medical simulation based on head movements of the trainee as sensed by the virtual reality headset 135. For example, if a user gazes at one user interface element in the VR scene for longer than 5 seconds, the gaze information can be sent to the medical simulation system. In other examples, if the quantity of anesthetics is changed on the monitoring system in the VR, or if the energy-level for an electro-cautery tool changes before applying it to the tissue, the medical simulation system can be sent this information such that the simulation can be modified.

In some embodiments, the trainee can be wearing VR/AR glove(s) (not shown) that can sense hand motions of the trainee. In these embodiments, the VR/AR tracking response module 111d can modify the medical simulation based on the sensed movement of the gloves.

The surgical procedure tracking module 111e can track a status of the medical procedure simulation and can provide surgical procedure status to be reported to the VR/AR simulation system 115. Surgical procedure status can include changes to the patient (e.g., avatar) during the medical procedure simulation. For example, if the trainee has inserted a tool in a way that causes the avatar's body (e.g., patient's body) to react (e.g., move, bleed and/or shiver), vital signs changes of the avatar, movement of the abdomen with response to the movement of the fetus inside such that an ultrasound view is changed, and/or energy tool can malfunction in mid-surgery such that a message is displayed in the VR scene.

The communication management module 111c can transmit information from the modules shown in the medical procedure simulation system 110 to the connection module 120. The communication management module 111c can transmit the information as soon as it's available or with a frequency.

In some embodiments, the medical procedure simulation system 110 include a voice recognition component to receive voice input from the user. For example, if a trainee states “select scalpel” the medical procedure simulation system 110 can receive that audio input, recognize the content of the audio input (e.g., via voice recognition techniques as are known in the art), and the medical procedure simulation system 110 can update the tool in current use as the left trocar entry location and/or remove the previous tool from the simulation. The VR/AR nurse avatar can repeat the tool name and location in its own voice, and the nurse avatar can be displayed to the trainee as obtaining the proper tool and bringing it to the trainee's VR hand or proper location on the patient (e.g., avatar's) body.

The VR simulation system 115 can be coupled to the virtual reality headset 135. The virtual reality headset can be virtual reality headsets as are known in the art. For example, the virtual reality headset can be an Oculus Rift, HTC Vive, or Samsung Gear VR. As is apparent to one of ordinary skill in the art, for embodiments, where the VR simulation system 115 only includes AR, the virtual reality headset 135 can be a AR headset only (e.g., AR glasses). For example, a Microsoft Hololens, or any AR reality headset as is known in the art.

The VR simulation system 115 can include an avatar head/hands movement module 116a, a VR/AR tracking response module 116b, a surgical procedure response module 116c, a tool handle movement render module 116d, a procedure distractions module 116e, a surgical tool selection module 116f, a vital signs module 116g, a patient behavior module 116h, a VR post effects module 116i, or any combination thereof. As is apparent to one of ordinary skill in the art, in embodiments where the VR simulation system 115 is an AR system, the VR post effects module 116i is an AR post effects module.

The VR/AR tracking response module 116b can cause the VR scene to respond to the head movements of the trainee as sensed by the virtual reality headset 135. For example, if the trainee turns their head to the left, the VR scene can show the left side of the operating room. If the trainee bends down towards the avatar (e.g., the patient) to, for example, see an incision on the patient more clearly, the VR scene can show the incision zoomed similar to what is experienced by a person in real life.

The surgical procedure response module 116c can receive surgical procedure status information from the medical procedure simulation system 111 (e.g., via the surgical procedure tracking module). The surgical procedure response module 116c can cause the VR scene to be modified according to the surgical procedure status. The surgical procedure response module 116c can include surgical status that is effected by the VR operating room scene. For example, if a second trainee knocks over a table onto an open wound of the avatar.

The surgical tool selection module 116f can receive surgical tool status information from the medical procedure simulation system 110. For example, [IGNORE: SIMBIONIX, PLEASE INSERT AN EXAMPLE]. The VR simulation system 115 can modify the VR/AR operating room scene based on the surgical tool status information. For example, the VR/AR simulation can render the surgical tool in the VR/AR scene at a location that correlates to the position of the surgical tool in the medical procedure simulation 110. The surgical tool selection module 116f can send status of virtual surgical tools to the medical procedure simulation system 110.

The procedure distractions module 116e can randomly activate distractions that can alter a trainee's behavior. For example, a surgeon trainee can be paged in the VR operating room scene, the OR door can open and staff member can pose a question to a bot on the operating team, some of the staff (e.g., bots or other participants) can start chatting and/or the nurse can provide a tool other than what was indicated.

The vital signs module 116g can modify the VR operating room scene based on vital sign information from the medical procedure simulation system 110. For example, the VR operating room scene can include one or more vital sign monitors which can display the vital sign information (e.g., pulse, temperature and/or oxygen level). The avatar's behavior can correspond to the vital signs. For example, in the case of an injury to a large vessel a sudden decrease in blood pressure can be displayed.

The patient behavior module 116h can modify the avatars visual appearance based on the surgical procedure status from the medical procedure simulation system 110. For example, the VR avatar can appear as bleeding, having palpitations and/or stomach deflation.

In some embodiments, a second trainee can participate in the simulation via a second system. In these embodiments, the VR/AR simulation system 110 can receive inputs and/or output information to the second system. The tool handle movement render module 116d can receive tool information from the second system and determine what tool information to display in the VR operating room scene.

The avatar head/hands movement module 116a can receive head and/or hand movement information from a second system and render that movement in the VR scene for the trainee of the first system.

The trainee and/or avatar within the VR/AR scene can be medical personnel, including nurses, doctors, physicians assistants, medical personnel related to certain procedures (e.g., a hip replacement manufacturer doctor that monitors hip replacement surgeries).

The VR post effects module 116i can add effects to surfaces rendered by the medical simulation module 110 and shared with the VR simulation system 115. For example, assume the medical procedure simulation module 110 provides the VR simulation system 115 with a rendering of vital signs of the bot during the simulation. Also assume that the VR scene is in a darkly lit room. The VR post effects module 1116i can add the post effect of lightening the vital signs rendering provided by the medical procedure simulation module 110. Other post effects can include noise effects (e.g., monitor malfunction), blur effects (e.g., camera malfunction).

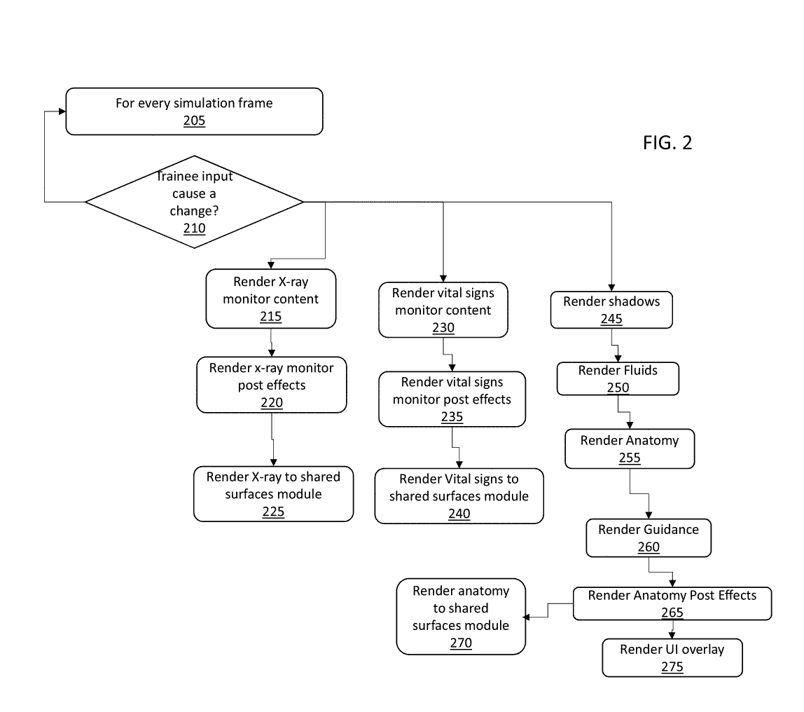

During operation, the medical procedure simulation module 110 can render the objects for the medical simulation. The rendering can be a two dimensional or three dimensional rendering, for example, as is known in the art. FIG. 2 is a flow chart for a method for rendering via the medical procedure simulation module of FIG. 1, according to an illustrative embodiment of the invention. The method can involve for every simulation frame (Step 205), determine if input of the trainee has caused a change in the medical procedure simulation (e.g., has the trainee moved the haptic tool and/or the VR glasses or gloves, such that the medical procedure is effected) (Step 210). If no change has been made, then continue to the next frame (e.g., back to step 205). If a change has been made then, the method can involve the remaining steps of FIG. 2.

The method can involve rendering an x-ray monitor content (Step 215). The method also involves rendering post effects for the x-ray monitor (Step 220). For example, grayscale and/or FXAA. The method also involves rendering the x-ray monitor content to the shared surfaces module (Step 225). The x-ray monitor can be rendered as being in the operating room, and displaying images of the x-ray taken during the simulated medical procedure.

The method can involve rendering vital signs monitor content (Step 230). The method also involves rendering post effects for the vital signs monitor (Step 235). The method also involves rendering the vital signs monitor content to the shared surfaces module. The vital signs monitor can be rendered as being in the operating room, and displaying images of the vital signs during the simulated medical procedure.

The method can also involve rendering shadows (Step 245). For example, all static and dynamic shadows both in the simulation and the operating room. The method can also involve rendering fluids (Step 250). For example, blood, bile and/or water.

The method can involve rendering anatomy (Step 255). The anatomy can be the anatomy of the medical simulation. The method can also involve rendering guidance (Step 260). The guidance can be a step-by-step tutorial with visual effect (e.g., stickers, arrows) that appear on the simulated anatomy or a nurse bot that can guide a trainee during a procedure. The method can also involve rendering the anatomy with post effects (Step 265). The post effects can include high dynamic range rendering, depth of field, fluids on the anatomy, FXAA, and/or a blur filter.

The method can also involve rendering the anatomy to the shared surfaces module (Step 270).

The method can also involve rendering a user interface overlay (Step 275). The user interface can be the user interface in the virtual reality operating room. The user interface can include indicators for selected tool types, selected energy mode, camera angle and/or pedal.

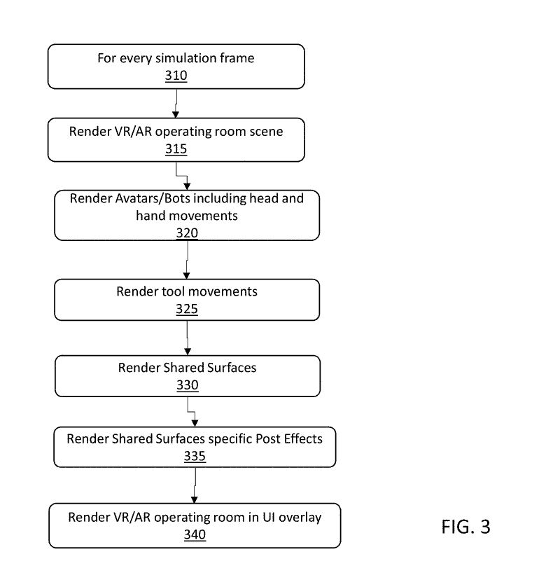

FIG. 3 is a flow chart for a method for rendering via the VR simulation system of FIG. 1. The method can involve for every simulation frame (Step 310), rendering a VR/AR operating room scene (Step 315). The method can also involve rendering one or more avatars (e.g., bots) in the operating room scene (Step 320). The avatars can be avatars that correlated to trainees on other simulations systems participating in the simulation or simulation generated avatars.

The rendering of the one or more avatars can include rendering head and hand movements of the one or more avatars in the operating room scene (Step 325). The head and hand movement can occur as a result of a particular occurrence in the OR. For example, in the case of an injury, the assistant can be moving its head towards the trainee, raising a hand and announcing an injury has occurred.

The method can also involve rendering movements of haptic tools in the operating room scene (Step 325). The method can also involve rendering surfaces from the shared surfaces module in the operating room scene (Step 330). The method can also involve rendering specific post effects on the shared surfaces in the operating room scene (Step 335)

The method can also involve rendering in VR/AR the operating room scene in a user interface overlay (Step 340).

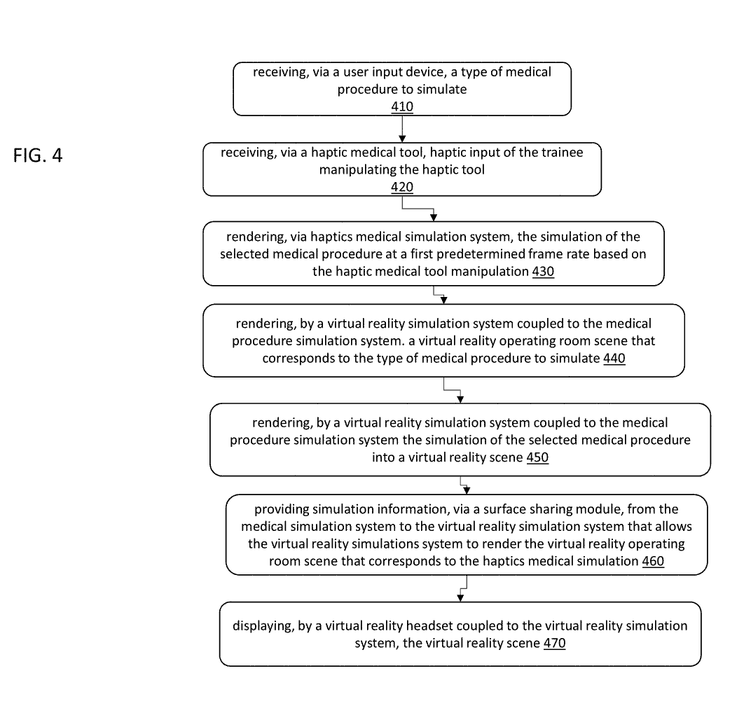

FIG. 4 shows a flow chart of a method for simulating medical procedures in a virtual reality operating room for training a trainee. The method involves receiving (e.g., via the input device 105 as described above in FIG. 1) a type of medical procedure to simulate (Step 410). The type of medical procedure can be specified by a trainee, a person who wants to monitor the trainee (e.g., teacher) or any other user. The type of medical procedure can be a surgery, diagnostic procedure using ultrasound and/or other imaging modalities, anesthesia, cardiovascular interventions, and/or emergency room treatments. The surgery can be on an infant, child and/or adult. In some embodiments, the surgery is on an animal. In some embodiments, once the medical procedure is specified, a virtual reality scene or augmented reality scene that corresponds to the medical procedure specified is rendered and presented to the trainee by a VR/AR headset.

The method can also involve receiving, via a haptic tool, (e.g., via the medical tool 125, as described above in FIG. 1) haptic input of the trainee manipulating the haptic tool (Step 420). The trainee can manipulate the haptic tool during the simulation to perform the simulated procedure.

The method can also involve rendering a simulation of the selected medical procedure, via a haptics medical simulation system, (e.g., via the medical procedure simulation system 110, as described above in FIG. 1) at a first predetermined frame rate based on the haptic medical tool manipulation (Step 430). The first predetermined frame rate can be based on desired performance of the simulated procedure. For example, for a highly responsive simulation the frame rate can be equal to that of the VR simulation frame rate. The first predetermined frame rate can have a minimum of 90 frames per second.

The rendering can also be based on the received type of medical procedure and the sensed motion and touch of the trainee. As the trainee moves the haptic tool, the simulation can receive the location and sensor information from the haptic tool. The simulation can interpret the movement and touch and render the simulation output based on that movement and touch. For example, a trainee can be operating on a simulated heart. If the trainee moves the tool slowly near an artery as shown in the VR/AR headset, the simulation can interpret that movement render the simulation as causing a slow cut in the heart.

The method can also involve rendering (e.g., via the VR/AR simulation system 115) a virtual reality operating room scene that corresponds to the type of medical procedure to simulate (Step 440). The virtual reality operating room can be rendered at a rate between 90 and 120 frames per second.

The method can also involve rendering (e.g., via the VR/AR simulation system 115) the simulation of the selected medical procedure into a virtual reality scene (Step 450). The simulation can be rendered onto an avatar in the VR/AR scene. The avatar can correspond to the type of procedure (e.g., child's bypass surgery).

The method can also involve providing simulation information, via a surface sharing module (e.g., the surface sharing module 122, as shown above in FIG. 1) from the haptics medical simulation system to the virtual reality simulation system to render the virtual reality operating room scene that corresponds to the haptics medical procedure simulation (Step 460). The simulation information can include x-ray information, ultrasound information, magnetic resonance imaging information, CT scan information, and/or other medical imaging as is known in the art. The simulation information can include anatomy, vital signs, and/or any combination thereof.

In some embodiments, the method involves modifying the simulation information as rendered by the virtual reality simulation system based on one or more post processing effects. In some embodiments, the one or more post processing effects are based on an environment in the virtual reality operating room scene. The environment can include, lighting with the virtual reality operating room scene, refraction and/or reflection.

The method can also involve displaying (e.g., via the virtual reality headset 135) the virtual reality scene (Step 470). In some embodiments, the method includes displaying via a AR headset a AR scene.

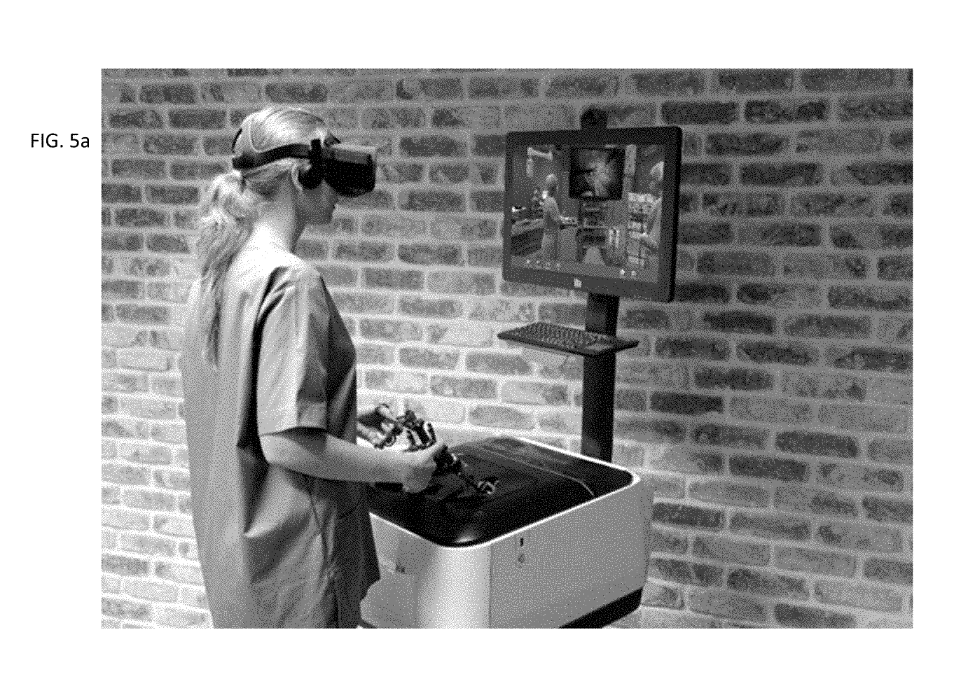

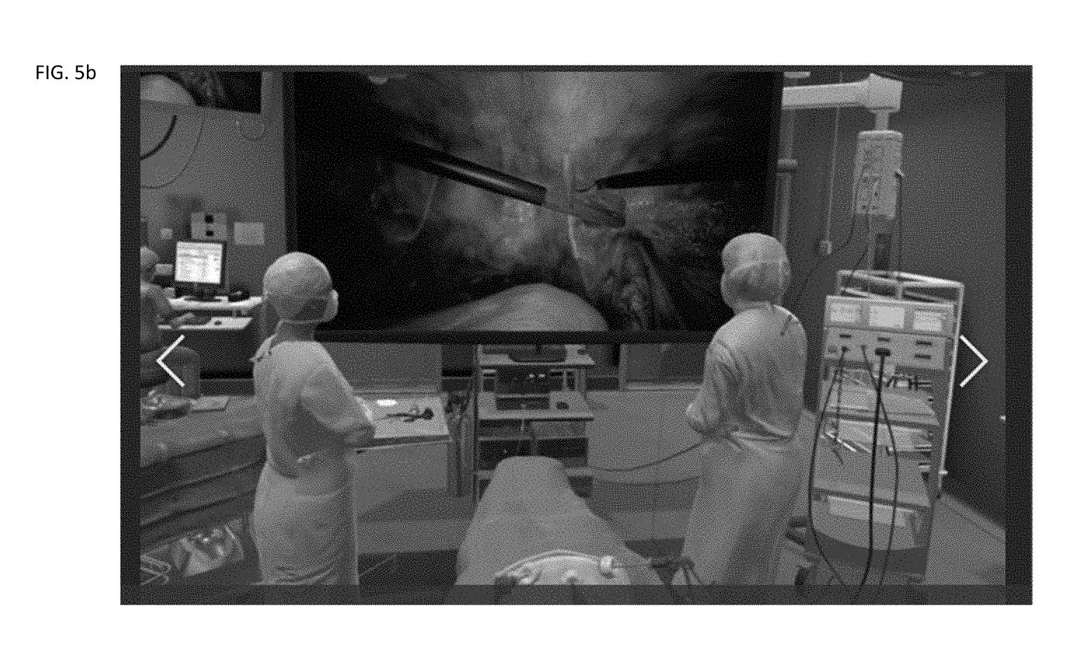

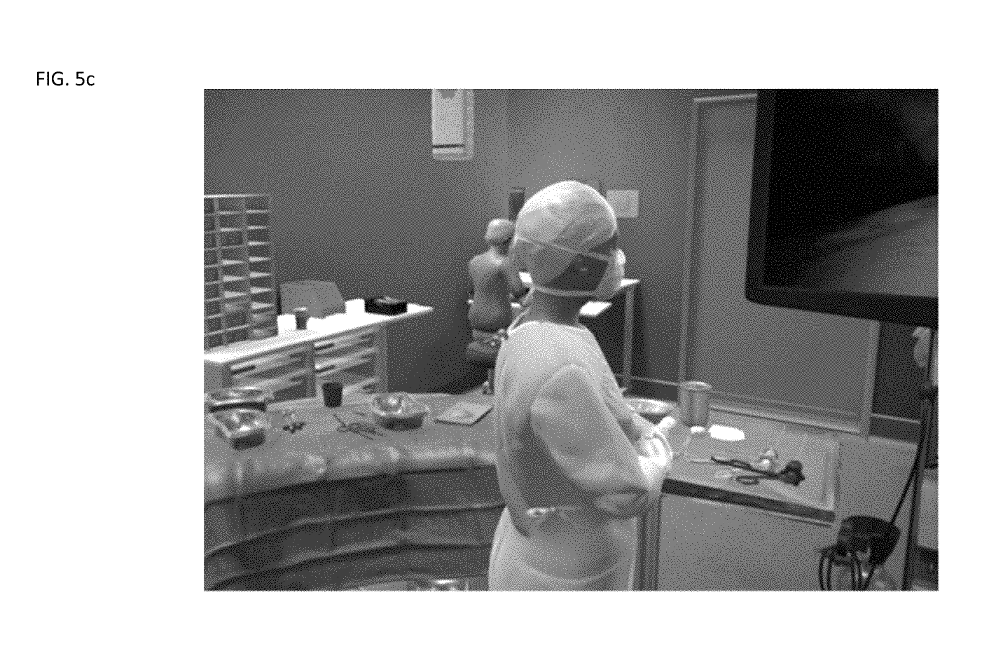

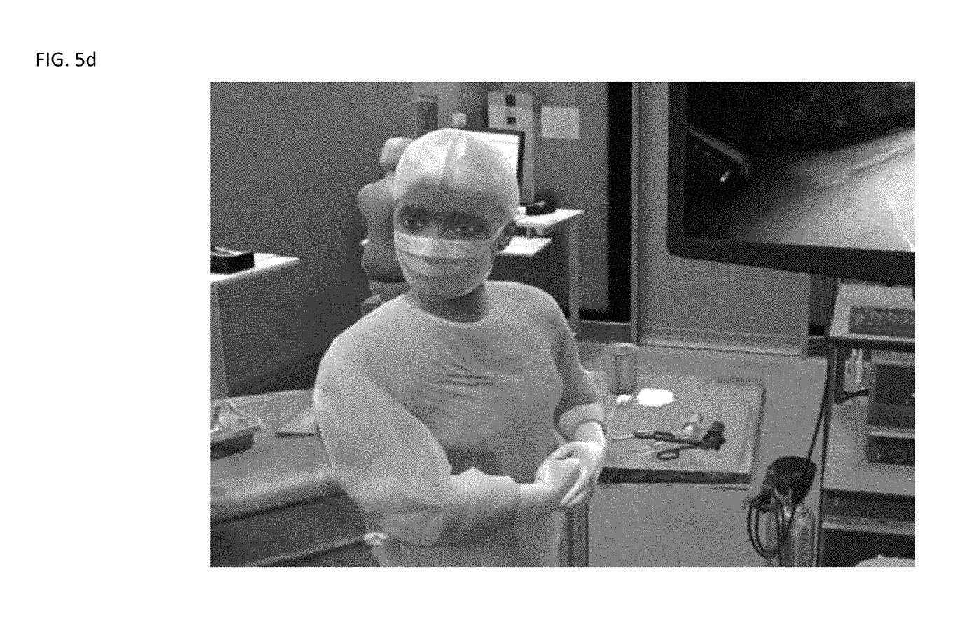

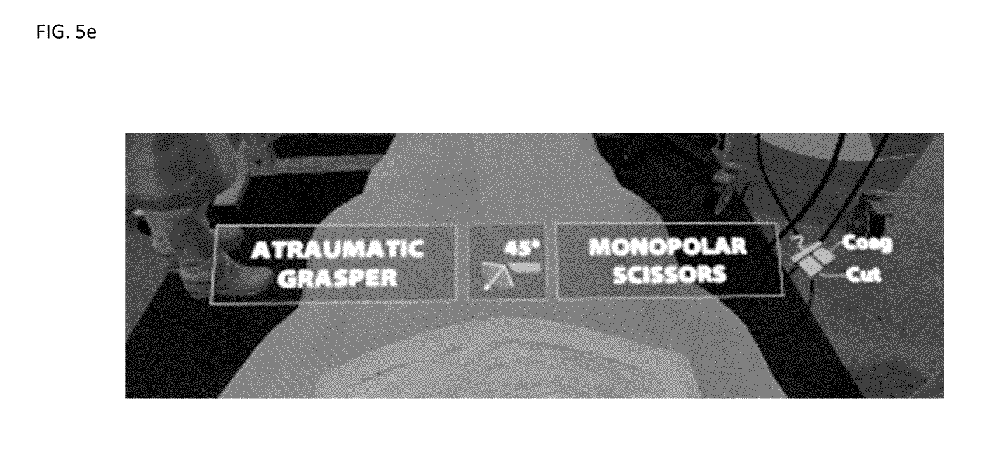

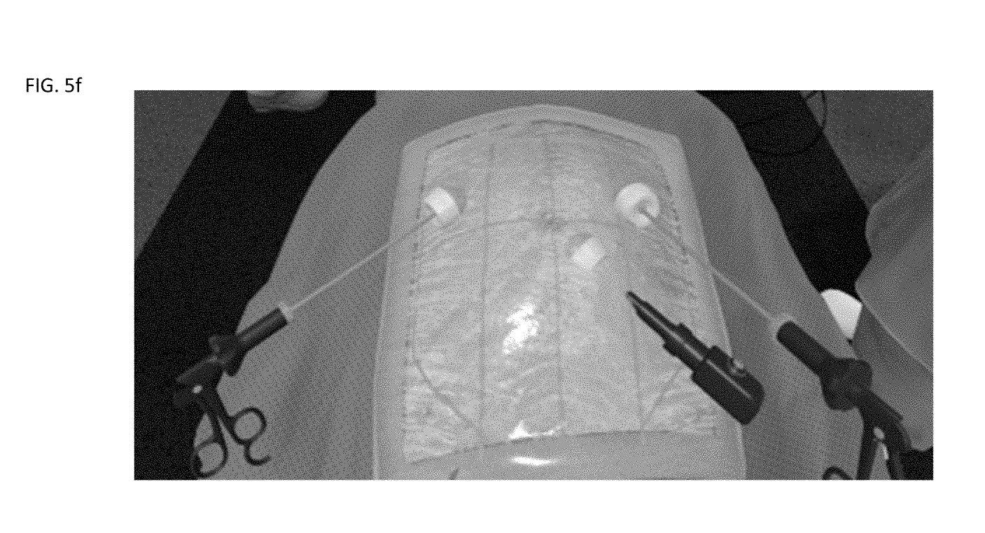

FIGS. 5a-5f are diagrams showing examples of a trainee using the simulation system of FIG. 1, 2 or the method of FIG. 3, according to illustrative embodiments of the invention. FIG. 5a shows an example of trainee holding two medical tools wearing a VR headset. Also shown is a two-dimensional screen showing a two-dimensional view of simulation. FIG. 5b shows an example of a two-dimensional screen shot of a virtual reality scene as viewed by the trainee in the virtual reality headset. The medical procedure simulation is shown on a screen, with two bots in the operating room. FIG. 5c shows an example of a screen shot of a virtual reality scene with a nurse bot in the operating room. FIG. 5d shows an example of a screen shot of a virtual reality scene with a nurse bot in the operating room talking to the trainee. FIG. 5e shows a screen shot of a user interface superimposed on the virtual reality scene. FIG. 5f shows an example of a screen shot a virtual reality scene with multiple tools for the trainee to use in virtual reality

The above-described methods can be implemented in digital electronic circuitry, in computer hardware, firmware, and/or software. The implementation can be as a computer program product (e.g., a computer program tangibly embodied in an information carrier). The implementation can, for example, be in a machine-readable storage device for execution by, or to control the operation of, data processing apparatus. The implementation can, for example, be a programmable processor, a computer, and/or multiple computers.

A computer program can be written in any form of programming language, including compiled and/or interpreted languages, and the computer program can be deployed in any form, including as a stand-alone program or as a subroutine, element, and/or other unit suitable for use in a computing environment. A computer program can be deployed to be executed on one computer or on multiple computers at one site.

Method steps can be performed by one or more programmable processors executing a computer program to perform functions of the invention by operating on input data and generating output. Method steps can also be performed by an apparatus and can be implemented as special purpose logic circuitry. The circuitry can, for example, be a FPGA (field programmable gate array) and/or an ASIC (application-specific integrated circuit). Modules, subroutines, and software agents can refer to portions of the computer program, the processor, the special circuitry, software, and/or hardware that implement that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer can be operatively coupled to receive data from and/or transfer data to one or more mass storage devices for storing data (e.g., magnetic, magneto-optical disks, or optical disks).

Data transmission and instructions can also occur over a communications network. Information carriers suitable for embodying computer program instructions and data include all forms of non-volatile memory, including by way of example semiconductor memory devices. The information carriers can, for example, be EPROM, EEPROM, flash memory devices, magnetic disks, internal hard disks, removable disks, magneto-optical disks, CD-ROM, and/or DVD-ROM disks. The processor and the memory can be supplemented by, and/or incorporated in special purpose logic circuitry.

To provide for interaction with a user, the above described techniques can be implemented on a computer having a display device, a transmitting device, and/or a computing device. The display device can be, for example, a cathode ray tube (CRT) and/or a liquid crystal display (LCD) monitor. The interaction with a user can be, for example, a display of information to the user and a keyboard and a pointing device (e.g., a mouse or a trackball) by which the user can provide input to the computer (e.g., interact with a user interface element). Other kinds of devices can be used to provide for interaction with a user. Other devices can be, for example, feedback provided to the user in any form of sensory feedback (e.g., visual feedback, auditory feedback, or tactile feedback). Input from the user can be, for example, received in any form, including acoustic, speech, and/or tactile input.

The computing device can include, for example, a computer, a computer with a browser device, a telephone, an IP phone, a mobile device (e.g., cellular phone, personal digital assistant (PDA) device, laptop computer, electronic mail device), and/or other communication devices. The computing device can be, for example, one or more computer servers. The computer servers can be, for example, part of a server farm. The browser device includes, for example, a computer (e.g., desktop computer, laptop computer, and tablet) with a World Wide Web browser (e.g., Microsoft® Internet Explorer® available from Microsoft Corporation, Chrome available from Google, Mozilla® Firefox available from Mozilla Corporation, Safari available from Apple). The mobile computing device includes, for example, a personal digital assistant (PDA).

Website and/or web pages can be provided, for example, through a network (e.g., Internet) using a web server. The web server can be, for example, a computer with a server module (e.g., Microsoft® Internet Information Services available from Microsoft Corporation, Apache Web Server available from Apache Software Foundation, Apache Tomcat Web Server available from Apache Software Foundation).

The storage module can be, for example, a random access memory (RAM) module, a read only memory (ROM) module, a computer hard drive, a memory card (e.g., universal serial bus (USB) flash drive, a secure digital (SD) flash card), a floppy disk, and/or any other data storage device. Information stored on a storage module can be maintained, for example, in a database (e.g., relational database system, flat database system) and/or any other logical information storage mechanism.

The above-described techniques can be implemented in a distributed computing system that includes a back-end component. The back-end component can, for example, be a data server, a middleware component, and/or an application server. The above described techniques can be implemented in a distributing computing system that includes a front-end component. The front-end component can, for example, be a client computer having a graphical user interface, a Web browser through which a user can interact with an example implementation, and/or other graphical user interfaces for a transmitting device. The components of the system can be interconnected by any form or medium of digital data communication (e.g., a communication network). Examples of communication networks include a local area network (LAN), a wide area network (WAN), the Internet, wired networks, and/or wireless networks.

The system can include clients and servers. A client and a server are generally remote from each other and typically interact through a communication network. The relationship of client and server arises by virtue of computer programs running on the respective computers and having a client-server relationship to each other.

The above described networks can be implemented in a packet-based network, a circuit-based network, and/or a combination of a packet-based network and a circuit-based network. Packet-based networks can include, for example, the Internet, a carrier internet protocol (IP) network (e.g., local area network (LAN), wide area network (WAN), campus area network (CAN), metropolitan area network (MAN), home area network (HAN), a private IP network, an IP private branch exchange (IPBX), a wireless network (e.g., radio access network (RAN), 802.11 network, 802.16 network, general packet radio service (GPRS) network, HiperLAN), and/or other packet-based networks. Circuit-based networks can include, for example, the public switched telephone network (PSTN), a private branch exchange (PBX), a wireless network (e.g., RAN, Bluetooth®, code-division multiple access (CDMA) network, time division multiple access (TDMA) network, global system for mobile communications (GSM) network), and/or other circuit-based networks.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the foregoing detailed description, numerous specific details are set forth in order to provide an understanding of the invention. However, it will be understood by those skilled in the art that the invention can be practiced without these specific details. In other instances, well-known methods, procedures, and components, modules, units and/or circuits have not been described in detail so as not to obscure the invention. Some features or elements described with respect to one embodiment can be combined with features or elements described with respect to other embodiments.

1. A system for rendering medical procedures in a virtual reality operating room for training a trainee, the system comprising:
a user input device for the trainee to select a type of medical procedure to simulate;
a haptic medical tool for the trainee to manually manipulate during the simulation;
a haptics medical simulation system for rendering the simulation of the selected medical procedure at a first predetermined frame rate based on the haptic medical tool manipulation;
virtual reality simulation system coupled to the medical procedure simulation system to render i) a virtual reality operating room scene that corresponds to the type of medical procedure to simulate, and ii) the simulation of the selected medical procedure into a virtual reality scene;
a surface sharing module coupled to the haptics medical simulation system and the virtual reality simulation system, the surface sharing module providing simulation information from the simulation system to the virtual reality simulation system that allows the virtual reality simulation system to render the virtual reality operating room scene that corresponds to the haptics medical simulation; and
a virtual reality headset coupled to the virtual reality simulation system for the trainee to view the virtual reality scene.

2. The system of claim 1 wherein the simulation information comprises x-ray, Ultrasound, magnetic resonance imaging, CT scan, monitor haptic simulation information, anatomy, vital signs or any combination thereof.

3. The system of claim 1 wherein the virtual reality simulation system modifies the simulation information from the surface sharing module with post processing effects.

4. The system of claim 3 wherein the post processing effects comprises modifying visual appearance of rendered object that correspond to the simulation information based on the environment in the virtual reality operating room scene.

5. The system of claim 1 wherein the virtual reality simulation system renders the virtual reality scene with a priority over the haptics medical simulation system.

6. The system of claim 1 wherein the virtual reality simulation system renders at a rate of at least 90 frames per second.

7. The system of claim 6 wherein the haptics medical simulation renders at a rate of the virtual reality simulation system.

8. The system of claim 1 wherein the haptics medical simulation system and the virtual reality simulation system are rendering the medical simulation in parallel.

9. A method for rendering medical procedures in a virtual reality operating room for training a trainee, the method comprising:
selecting a medical procedure to simulate a user input device for the trainee to select a type of medical procedure to simulate;
receiving haptic input from a haptic medical tool for the trainee to manually manipulate during the simulation;
rendering, via a haptics medical simulation system, the simulation of the selected medical procedure at a first predetermined frame rate based on the haptic medical tool manipulation;
rendering, via a virtual reality simulation system coupled to the medical procedure simulation system, i) a virtual reality operating room scene that corresponds to the type of medical procedure to simulate, and ii) the simulation of the selected medical procedure into a virtual reality scene;
providing simulation information, via a surface sharing module, from the medical simulation system to the virtual reality simulation system that allows the virtual reality simulation system to render the virtual reality operating room scene that corresponds to the haptics medical simulation; and
displaying, via a virtual reality headset, the virtual reality scene.

10. The method of claim 9 wherein the simulation information comprises x-ray, Ultrasound, magnetic resonance imaging, CT scan, monitor haptic simulation information, anatomy, vital signs or any combination thereof.

11. The method of claim 9 further comprising modifying, via the virtual reality simulation system, the simulation information from the surface sharing module with post processing effects.

12. The method of claim 11 wherein the post processing effects comprises modifying visual appearance of rendered object that correspond to the simulation information based on the environment in the virtual reality operating room scene.

13. The method of claim 9 wherein the virtual reality simulation system renders the virtual reality scene with a priority over the haptics medical simulation system.

14. The method of claim 9 wherein the virtual reality simulation system renders at a rate of at least 90 frames per second.

15. The method of claim 14 wherein the haptics medical simulation renders at a rate of the virtual reality simulation system.

16. The method of claim 9 wherein the haptics medical simulation system and the virtual reality simulation system are rendering the medical simulation in parallel.
